(19) United States
(12) Patent Application Publication
(10) Pub. No.: US 2023/0190440 A1
(43) Pub. Date: Jun. 22, 2023

(54) Novel Elastomer Gel Liner with Perspiration Elimination Mechanism

(71) Applicants: Aldo Laghi, Pinellas Park (CZ); Alps South Europe, Plzen (CZ)

(72) Inventor: Aldo Laghi, Pinellas Park, FL (US)

(21) Appl. No.: 18/113,662

(22) Filed: Feb. 24, 2023

Related U.S. Application Data
(63) Continuation-in-part of application No. 18/083,658, filed on Dec. 19, 2022.
Application No. 18/083,658 is a continuation-in-part of application No. 17/742,721, filed on May 12, 2022.
Application No. 17/742,721 is a continuation-in-part of application No. 16/868,776, filed on May 7, 2020, now Pat. No. 11,602,445.

(51) Int. Cl. A61F 2/00; A61F 2/02

(52) U.S. Cl. CPC A61F 2/0077; A61F 2/02; A61F 2002/30067

(57) ABSTRACT

A method of making a prosthetic liner for use with a prosthetic assembly that acts as the interface between the residual limb of an amputee and the socket assembly. The prosthetic liner comprises an open proximal end, a closed distal end, and sidewalls comprising an inner layer of molded thermoplastic gel. A thermoplastic material is molded over a mandrel that has been sandblasted using #36 grit and, optionally, #320 grit at 100 psi so as to form microcraters and reduce the coefficient of static friction. The liner further includes a sweat port incorporated into the distal end with a one-way valve for the elimination of perspiration.

### CROSS-REFERENCE TO RELATED APPLICATIONS

This application is a continuation-in-part of application Ser. No. 18/083,658 entitled “Novel Elastomeric Gel Liner”, filed Dec. 19, 2022, which is a continuation-in-part of application Ser. No. 17/742,721 entitled “Novel Silicone Liner”, filed May 12, 2022, which is a continuation-in-part of application Ser. No. 16/868,776 entitled “Novel Silicone Liner”, filed May 7, 2020, which claimed the benefit of provisional application No. 62/844,253, filed May 7, 2019, and provisional application No. 62/844,296, filed May 7, 2019, the disclosures of which are incorporated by reference herein.

### BACKGROUND OF THE INVENTION

### Field of the Invention

This invention relates to liners for use in a prosthetic assembly. Specifically, the described invention relates to liners having a thermoplastic elastomer lining that is smooth to the touch via the use of surface modification of the molds used to create the liners and which are designed to relieve perspiration between the liner and the residual limb of the wearer.

### Description of the Background Art

Silicone liners have been used since the 1980s in the prosthetic industry such as those described in U.S. Pat. No. 4,923,474 granted to Klasson and Kristinsson. Other examples of such liners include U.S. Pat. No. 5,728,168 to Laghi et al., U.S. Pat. No. 5,830,237 granted to Kania, U.S. Pat. No. 5,507,834 to Laghi et al., U.S. Pat. No. 5,443,525 to Laghi et al., and U.S. Pat. No. 5,728,168 to Laghi et al.

However, silicone liners have historically been difficult to don and doff due to the high coefficient of static friction of silicone. As such, they tend to stick to the skin of a residual limb. This prevents relative movement at the interface skin/liner and therefore induces high shear forces on the skin in localized portions of the residual limb as the ground reaction to ambulation is transmitted to the skeleton through the silicone interface and the skin. These resulting shear forces increase the likelihood of blistering of the skin, especially for patients with sensitive skin. It is therefore desired to develop a silicone liner having a reduced frictional effect such that the user can easily don and doff their prosthetic liner.

Further, most amputees have had amputations for vascular, as opposed to traumatic, reasons. This means that the amputation was because of poor circulation. Most amputees are also elderly. As such, most amputees have thinner, more delicate, skin which is prone to damage and have reduced blood flow to the extremities giving them less ability to heal sores and wounds. Some elderly amputees end up having recurring amputations as the skin of their residual limbs becomes damaged and infected.

One method that has been developed to thwart these issues is to include additives that exude from the silicone matrix and act as a lubricant between the liner and skin. The issue with this solution is that the exudate can collect dirt on the inside surface of the liner, exacerbating skin damage. Exudates also require more thorough cleaning and make the liner slippery.

Likewise, it is known in the art to use a thermoplastic elastomer gel instead of silicone for prosthetic liners such as those described in 11,179,252 to Laghi et al. These thermoplastic elastomer gels generally comprise a styrene triblock copolymer mixed with a plasticizer which forms a composite with an outer fabric layer. One of the issues with gel liners is that they can be considered sticky by certain users which can prevent relative movement at the interface between the skin and liner.

The present invention utilizes a novel surface modification technique so as to create “microcraters” in the silicone or elastomer gel used for the prosthetic liner, as it is molded, which allow for the liner to be donned and doffed with ease and which is skin-friendly. The method described herein is particularly effective for silicone liners because silicones, when in the liquid state, have low surface tension which allows them to penetrate tiny holes and cracks and, therefore, to create the microcraters. Silicones' viscosity is also inversely proportional to temperature which makes silicone flow easier as they get closer to the surface of the hot mold. As a result, silicones produce a faithful mirror image of the cratered mold surface. Despite having different molding processes, the elastomeric gels normally used for prosthetic liners share similar properties when using the methods described herein.

One issue with elastomeric liners is their ability to thermally insulate which tends to increase the rate at which the residual limb perspires. The supple elasticity which makes elastomeric liners suitable for long term skin contact can have negative consequences when the contact with the skin is broken by a liquid, such as perspiration. While mild perspiration may enhance the seal between the residual limb and the liner, the amount produced quickly increases such that a layer of perspiration is formed between the liner surface and the residual limb surface.

Because of the pressures exerted on the liner during ambulation, particularly by the prosthetic socket, the liner can pull away from the residual limb, causing air to be sucked into the liner. As the air pockets join upon further ambulation, a volume is produced between the liner and the limb. The air in the volume contracts and expands with each step, creating a suction and causing the residual limb to expand inside the liner. Such an expansion affects the fit of the limb and liner inside the socket. Many methods for fitting the limb inside the prosthetic socket can require that the limb be repositioned in order to give a comfortable fit. However, upon cessation of ambulation, such as while sitting down or sleeping, it is not uncommon for the residual limb to shrink inside the liner to its original size, necessitating yet another refitting. Thus, it is of paramount importance that air be excluded from the liner. Toward that end, it is thus important that the build-up of perspiration be prevented, and that perspiration be removed as it is being formed, or soon thereafter, from between the liner and the residual limb. It would be an advance in the art to provide a liner having the beneficial advances of a microcratered surface in addition to the ability to eliminate perspiration.

Therefore, it is an object of this invention to provide an improvement which overcomes the aforementioned inadequacies of the prior art devices and provides an improvement which is a significant contribution to the advancement of the liner art.

Another object of the invention is to provide a silicone liner with a more comfortable interior silicone lining.

Another object of the invention is to provide a method of manufacturing silicone liners having reduced frictional characteristics.

Another object of the invention is to provide a liner having microcraters.

Another object of the invention is to describe a method of making silicone liners by molding the liners using mold mandrels that have been sandblasted.

Another object of the invention is to provide a method of manufacturing a thermoplastic elastomeric gel liner having reduced frictional characteristics.

Another object of the invention is to provide a prosthetic liner having a perspiration voiding system which functions upon ambulation with or without a vacuum pump.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to a liner for use in a prosthetic assembly having a thermoplastic elastomer gel interior comprising microcraters by sandblasting the mandrel using #36 grit at 100 psi. Using a sandblasted mold mandrel, a prosthetic liner can be manufactured having improved friction characteristics. The liner of the present invention may optionally include a fabric cover bonded to the exterior surface. The liner further includes a sweat port incorporated into the distal end with a one-way valve for the elimination of perspiration.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a front view of the improved prosthetic liner;

FIG. 2 is a sectional view of a silicone or thermoplastic gel sheet formed using a mandrel that has not been sandblasted;

FIG. 3 is a sectional view of a silicone or thermoplastic gel sheet formed using a mandrel sandblasted with #36 grit at 100 psi;

FIG. 4 is a sectional view of a silicone or thermoplastic gel sheet formed using a mandrel sandblasted with #320 grit at 100 psi;

FIG. 5 is a graph showing the effect of sandblasting on the pulling force necessary to remove a silicone sheet from a steel substrate showing the effect on the static coefficient of friction;

FIG. 6 is a cross-sectional view of a non-pin locking liner incorporating a perspiration elimination mechanism of the present invention;

FIG. 7 is a cross-sectional view of the pin-locking liner incorporating a perspiration elimination mechanism of the present invention;

FIG. 8 is a cross-sectional view of an embodiment of the present invention wherein a buttress is present having cylindrically symmetric sections;

FIG. 9 is an elevational view of an integral sweat port and pin combination;

FIG. 10A is a top view of a separate sweat port having a vent; and

FIG. 10B is a side view of the sweat port illustrated in FIG. 10A.

Similar reference numerals refer to similar parts throughout the several views of the drawings.

### DETAILED DESCRIPTION OF THE PREFERRED EMBODIMENT

The following description is of the best mode presently contemplated for carrying out the invention. This description is not to be taken in a limiting sense, but is made merely for the purpose of describing one or more preferred embodiments of the invention. The scope of the invention should be determined with reference to the claims.

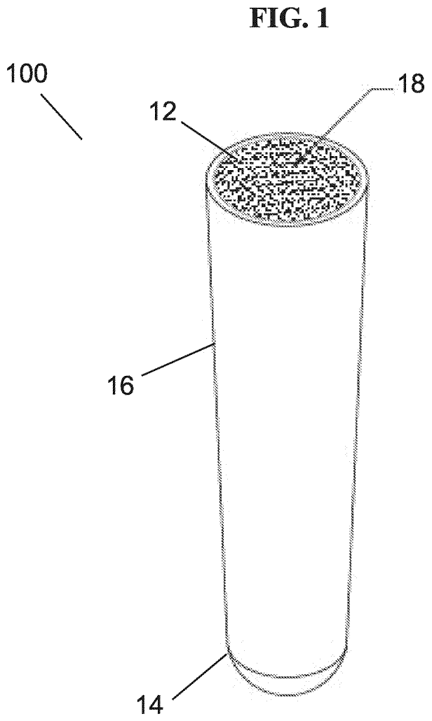

The present invention relates to a liner 100 for use with prosthetic devices. As shown in FIG. 1, the liner 100 for use with a prosthetic assembly comprises an open upper end 12 for receiving a residual limb, not shown, a closed bottom end 14, and sidewalls 16 of predetermined thickness. The liner is airtight when donned over a residual limb. The preferred thickness of the sidewalls 16 is about 1.5 mm to 3.0 mm. Note that the thickness is greater at the bottom end than in the sidewalls; the preferred thickness of the silicone at said bottom end 14 is about 3.0 mm to 12.0 mm. The sidewalls 16 have an inner layer 18 of the improved silicone or elastomer described herein. The sidewalls 16 can be fabric or another layer of more durable and higher friction silicone.

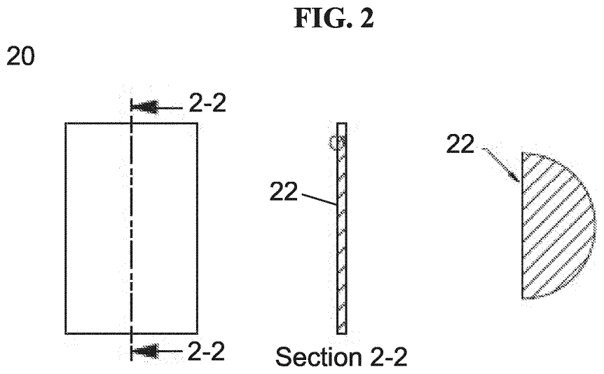

Prior to molding the silicone, a mandrel is sandblasted using #36 grit at 100 psi. The particular grit and pressure used provide the beneficial characteristics of the present invention. Other grits and pressures did not produce the benefits of reduced frictional hold and less chance of skin irritation. After sandblasting the mandrel, the silicone is molded over it, allowing the silicone to seep into the microcraters formed by the sandblasting. As can be seen in FIGS. 2-4, the size of grit used during sandblasting has a sizeable effect.

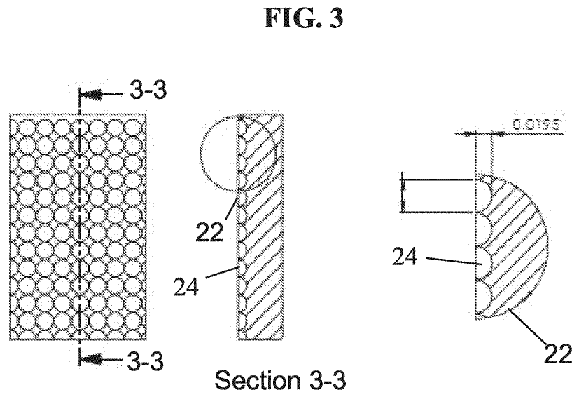

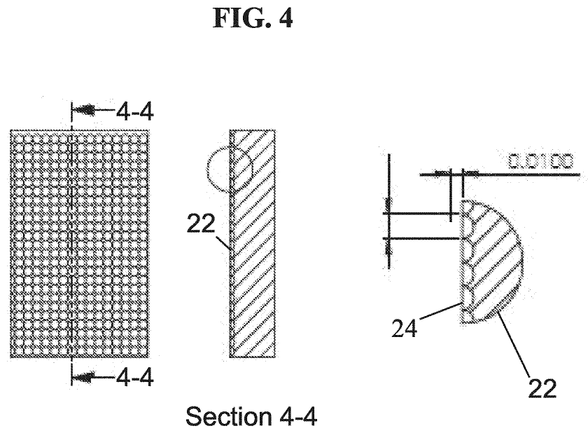

FIG. 2 depicts a sheet of silicone 20 that has not been sandblasted. As can be seen by the cross-section 2-2, not sandblasting the mandrel results in a smooth exterior surface 22 which maintains the high frictional characteristics of silicone. FIGS. 3 and 4, on the other hand, show a microscopic view of exterior surface 22 after having the mandrel sandblasted. FIG. 3 shows the microcratering the exterior surface 22 is subjected to using #36 grit at 100 psi thereby creating microcraters 24. Along line 3-3, the microcraters created generally have a depth of about 0.0195 mm. FIG. 4 shows the microcratering the exterior surface 22 is subjected to using #320 grit at 100 psi. Sandblasting with #320 grit may be done alone or after the mandrel has been sandblasted with #36 grit. Along line 4-4, the microcraters created generally have a depth of about 0.0100 mm. The deeper microcraters create a lower static coefficient of friction for silicone because there is less surface area for the exterior surface 22 to be in contact with, as can be seen when comparing the sheet in FIG. 3 with the sheet in FIG. 4. Sandblasting between #36 and #320 grit may also be performed.

Alternative methods of creating the microcraters 24 of differing depths include, but are not limited to, modification of the mandrel surface via rolling, compression of the mold against a textured surface, chemical etching, laser engraving, computer numerical control (CNC) engraving, electro-erosion (i.e. electrical discharge machining), electrodeposition, laser micro melting, shot blasting, shot peening, and pinwheeling. Similar methods now known or to be discovered which are equivalent to the methods included (i.e. methods that create microcraters in the exterior surface of a silicone layer in a prosthetic liner) are intended to be included in the above listing.

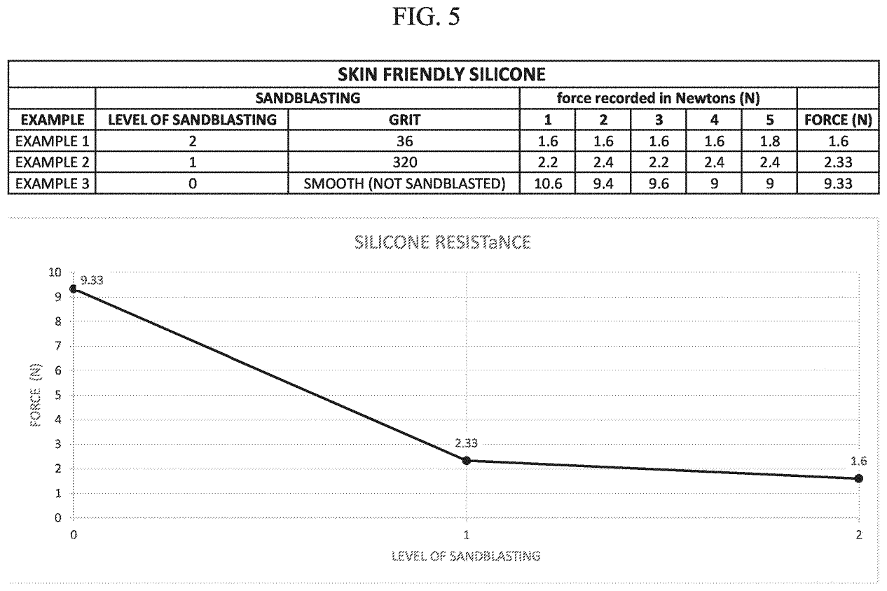

Three tests were performed to exhibit the beneficial properties of the present invention, the results of which are shown in FIG. 5. In the first, the mold was sandblasted using #36 grit at 100 psi and it took 1.6 N of force in terms of pull resistance, i.e. prior to tearing. In the second test, the mold was sandblasted using #320 grit. The result was 2.33 N of force in terms of pull resistance. In the final test the mold was not sandblasted at all and required 9.33 N of force. The tests were performed on smooth stainless steel using silicone strips that were 1-inch-wide and 7 inches long while applying 100 grams of weight.

The present invention can also be modified to improve the functionality of thermoplastic elastomer gel liners. Using thermoplastic molding techniques such as pressure molding or compression molding in combination with the methods described herein, the inner layer 18 of the liner 100 can be made of microcratered thermoplastic elastomer gel comprising a styrene triblock copolymer (such as those as described in U.S. Pat. No. 6,552,109 to Chen), polyurethane, polybutylene, or polypropylene. Unlike silicone, the sidewalls 16 of gel liners are usually thicker and can range from 3 to 12 millimeters in thickness with a preferred thickness at the bottom end 14 between 3 to 15 millimeters.

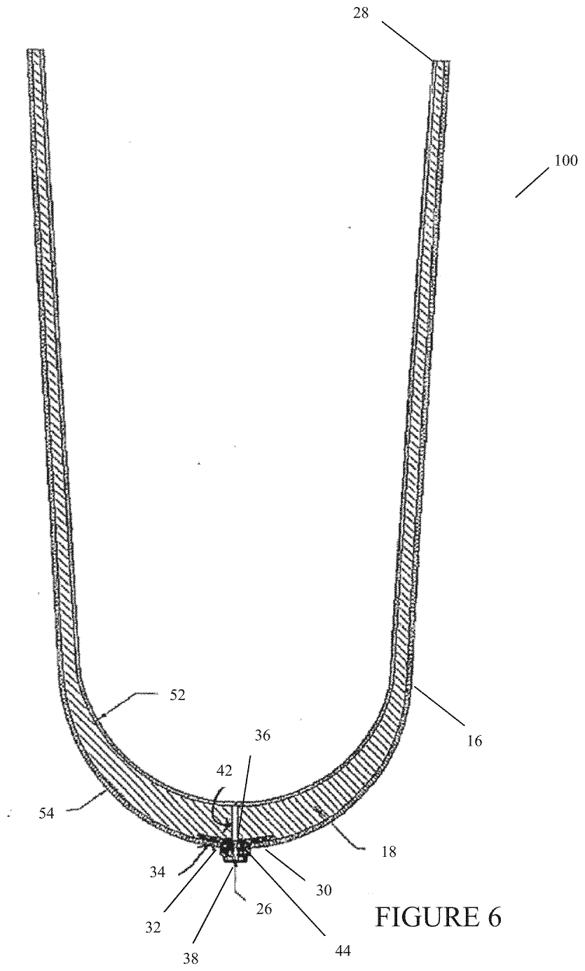

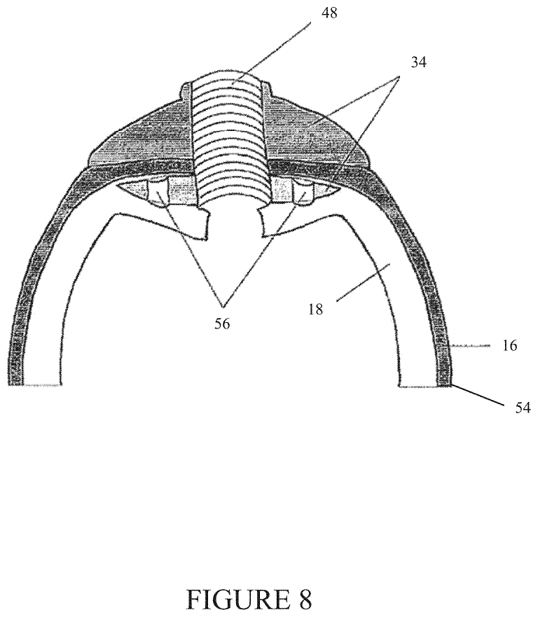

In addition, as shown in FIGS. 6-10B, the liner 100 may also include a sweat port 26. The liner 100 comprises a proximal edge 28 and a distal end 30 which includes a distal tip 32. A sweat port 26 is positioned at the distal tip 32. The distal tip 32 comprises, within it or attached to it, a buttress 34. The buttress 34 generally comprises a polymeric material having a degree of elasticity which is less than that of the liner, or in other embodiments, the buttress 34 comprises a support material, such as aluminum or other metals; or rigid plastics or other materials, which are over laid with a polymeric material. In embodiments in which the buttress 34 contacts the prosthetic socket, the polymeric component of the buttress softens the contact and prevents grinding which can wear the socket and irritate the wearer. The buttress can be embedded within the liner during liner manufacture, or in other embodiments, it is adhered to the end of the liner. It is preferably underlain by a section of the liner, regardless of whether it is embedded within the liner during liner manufacture or it is adhered to the liner distal tip during or after manufacture. The buttress 34 generally has cylindrical symmetry about the long axis of the liner. In one embodiment it is a disc of a thickness less than liner thickness and is entirely embedded within the liner. In another embodiment, it is partially within the liner and partially outside the liner (“anchored” in the liner). This embodiment is particularly useful in situations where a large buttress which is thicker than the liner is required.

In one embodiment, the buttress 34 is of a form such that a distal tip portion of the liner elastomer or silicone is between sections of buttress having cylindrical symmetry. In one embodiment, an internal buttress section 36 is embedded in the distal tip of the liner, and an external buttress section 38 is externally attached to the elastomeric liner distal tip. Such an arrangement can be created by mechanically sealing a portion of the distal tip section between the two buttress components, or it can be achieved by casting the liner around the buttress components using flow through holes 56, or otherwise including the buttress components in the fabrication and cooling of the liner, such that the buttress sections are essentially melt-sealed on both the inside and the outside of the distal tip of the liner.

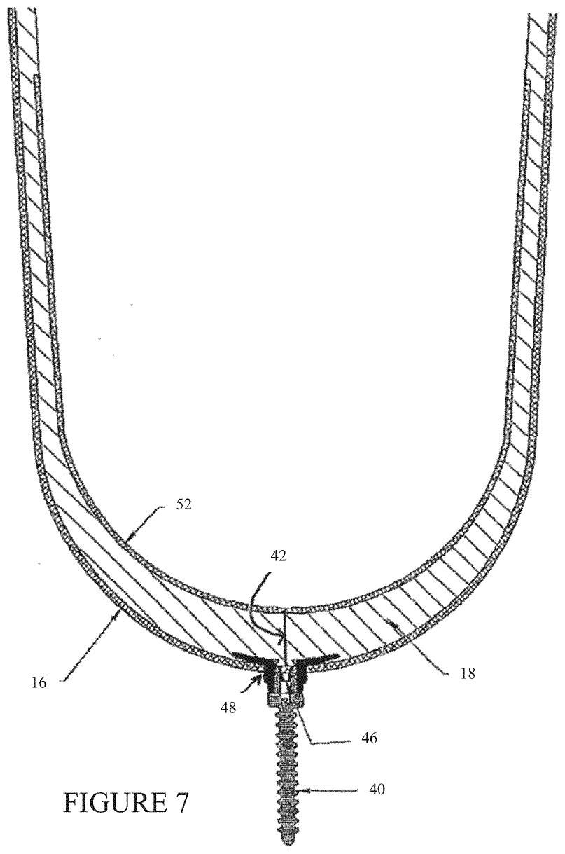

When the liner is in use, the buttress 34 is structurally sandwiched between the residual limb stump and the socket, providing critical support for the pin 40, as shown in FIG. 7. In some embodiments, the support is further improved in that the buttress is shaped to facilitate insertion into the socket, or the socket has a shape which complements the shape of the buttress. In preferred embodiments, the buttress extends at least 0.5 cm from the vertical axis of the liner, and is at least half the thickness of the liner body at its distal tip, and preferably, at least as thick as the liner body at its distal tip.

The inner layer 18 which can be used in the system of the present invention comprises an elastomeric material preferably of a type compatible with long periods of dynamic wearer contact. Such materials are known in art and may include the following polymer types and materials which include them: polyurethanes; block copolymers such as styrene block copolymers, generally non-limiting examples of which may include SEBS, SEPS, SEEBS and SEEPS and other types of styrene block copolymers. Further examples of styrene block copolymers which may be useful in the liner of the present invention include the so-called “controlled distribution” polymers, such as those disclosed in U.S. Pat. No. 7,226,484; U.S. Patent Application Publication Nos. 20070238835; 20050008669. Other potentially useful polymers may include so-called “crystalline” polymers, such as, for example, polymers disclosed in U.S. Pat. Nos. 5,953,396; 6,420,475; and 6,148,830. The above list is non-limiting, and in general, the list of acceptable polymers and gels includes those known in the art to be useful in the fabrication of prosthetic liners. By the term “gel,” it is meant a polymer having associated with it, through means known in the art, such as absorption, mixing or other, a plasticizer. A suitable liner which can be modified to include a buttress is the “EZ Gel” liner, available from Alps South L.L.C.

The inner layer 18 comprises a channel 42 passing through the liner from its inner surface to its outer surface. The channel 42 may be formed by piercing or punching, with or without the removal of elastomeric material from the liner. In one embodiment, the liner 100 is pierced such that little or no material is displaced, and the channel 42 is essentially closed by the elastomeric response of the liner material. In such cases, the pressures associated with the ambulatory motion of the patient can cause perspiration produced during wear to be squeezed through the channel 42. The channel 42 essentially acts as a relief valve which allows the elimination of perspiration emitted by the wearer. The perspiration occupies a volume between the liner and the wearer, potentially giving rise to undesirable interruption of vacuum. In the foregoing embodiment, a closed channel can assist in maintaining and reestablishing the vacuum.

The channel 42 can also be open. Such a situation is experienced with removal or displacement of liner material, such as with a punch or heated awl-type tool. As the buttress 34 sections extend over the distal tip 32, the channel 42 traverses them such that it opens out onto the distal tip 32 i.e., the portions of the channel 42 traversing the elastomer and those traversing the buttress sections are continuous. It should be noted that the channels through the elastomer and those through the buttress sections need not be of the same diameter. For example, it is permissible for the channel through the elastomer to be of the type described above in which perspiration can be passed under pressure.

The distal tip 32 of the liner, including the buttress 34, comprises a sweat port 26. The sweat port 26 is continuous with the channel 42, and it is preferably located approximately at the distal apex 44. By “distal apex” it is meant the lowest point on the long axis of the liner, including the buttress, when its long axis is oriented vertically with its distal tip pointing downward. As explained in greater detail infra, the sweat port 26 is attached to a pin which is received by the socket of the prosthetic. In some embodiments, the pin may not be precisely at the distal tip. For example, it may be desirable in some embodiments to attach the prosthetic in such a way that the pin is located at a point near, but not precisely at the distal apex. However, it should be noted that regardless of where the sweat port 26 is located, it is continuous with the channel 42. The sweat port 26 is a nipple or port extending from or partially/fully embedded or recessed in the distal tip 32 of the liner. In a preferred embodiment, at least a portion of the channel 42 through the buttress 34 contains threads 48, and the sweat port comprises matching threads 46 by which it can be screwably attached to the channel. In a preferred embodiment depicted in FIG. 6, the channel 42 extends from the surface of the distal tip 32 in a threaded extension which is attached to the liner buttress 34. In the embodiment depicted in FIG. 7, the threaded channel 42 is recessed in the distal tip surface of the liner buttress.

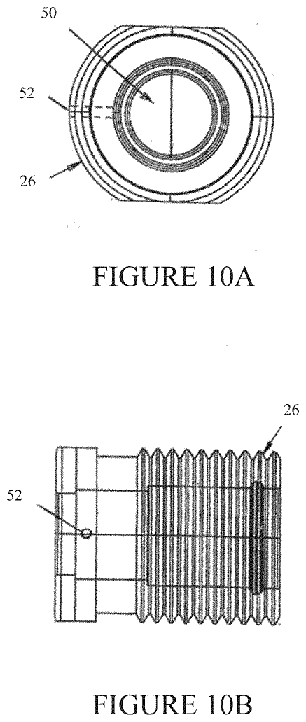

The sweat port 26, as shown in FIGS. 10A and 10B, contains a one-way valve which allows an exit for perspiration, air, and other liquids or gases which cause a volume increase between the liner and the wearer. The valve 50 is small enough to fit within the sweat port and can be one of many different kinds of check valves. In different embodiments, the valve is a duckbill, ball, diaphragm, swing, clapper, lift or other type of check valve. In a preferred embodiment, the one-way valve is a duck bill valve.

The sweat port 26 is attached to a pin 40 which, in use, is inserted into a prosthetic limb. The pin 40 is a spike of a durable material such as stainless steel or other metal or could be plastic, which is generally in the range of from about 1 inch to about 3 inches, which is inserted into a prosthetic limb. It can bear protrusions or other irregularities in profile which aid in securely fitting the prosthetic to the liner. Optionally, the prosthetic limb can include a locking mechanism which maintains the prosthetic securely in place.

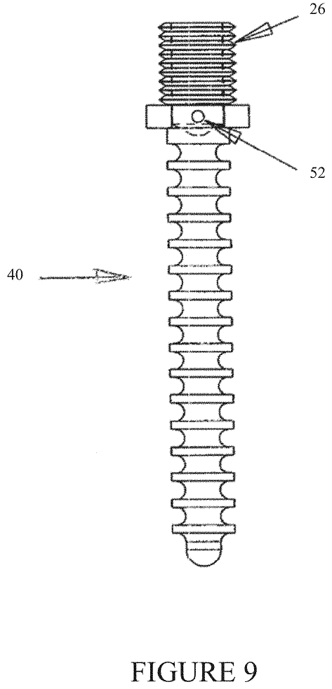

The sweat port 26 can be removably attached in series with the pin 40 as illustrated in FIG. 7 to form a port/pin assembly. Such a connection can be a threaded connection or other method of sealably engaging the sweat port 26 and the pin 40 and generally includes a vent 52 for the efflux of perspiration or another means of dissipating and draining the perspiration passing from the interior of the liner. In a preferred embodiment of the present invention, the sweat port 26 is integral with the pin 40, depicted in FIG. 9, for example, where a vent 52 is clearly visible on the side of the assembly.

In another embodiment of the present invention, the inner layer 18 has an inner fabric liner 52 on its inner surface which over lays the distal tip 32, and in some embodiments extends along the inner surface a distance in the range of from about 1 to about 19 inches from the proximal edge of the liner. In some embodiments, the fabric liner 52 extends to within no less than 0.5 inches from the proximal edge 28 of the liner 100, and in other embodiments, to within no less than 1.0 inches from the proximal edge 28. A fabric which absorbs perspiration helps to wick it toward the distal end of the liner. Preferred fabrics are those that absorb emitted perspiration, such as natural fibers, such as, for example, felt, wool and cotton fabrics and materials, as well as synthetic fabrics and materials, such as, for example rayon, orlon and nylon. In other embodiments, a sock which has a high perspiration absorbency can function as the fabric liner, preferably extending to no less than 0.5 inches from the proximal edge. It is preferred that the fabric have a thickness in the range of from about 0.5 to about 3.0 millimeters. It should be noted that regardless of fabric type and thickness, the present invention includes within its ambit embodiments comprising fabrics or materials having the ability to wick and/or store perspiration such that when weight is placed on the limb, the residual limb compresses the perspiration bearing fabric or material, and some or all of the perspiration is forced through the channel and the sweat port.

Furthermore, the fabric liner can be an elastic fabric to facilitate its ability to stretch somewhat with the motion of the liner. In some embodiments, the fabric liner comprises fabrics which are manufactured in such a way to optimize their elastic properties, especially when used as a liner material for an elastomeric liner body.

A benefit of the present invention is that the constant elimination of perspiration through the sweat port 26 one-way valve aids in restoring a vacuum to the liner/residual limb complex. A further benefit is that as small amounts of sweat are constantly eliminated through the sweat port, they evaporate readily from the area surrounding the sweat port, and thus no need exists to dispose of larger amounts of perspiration. However, if desired, perspiration can be routed to a receptacle, such as a void in the prosthetic or another method of containment.

In general, even in the absence of fabric, the compression motion which occurs during ambulation acts to force accumulated perspiration through the channel into the sweat port 26, and ultimately through the one-way valve 50. In this way, emitted perspiration is voided before it can have a significant effect on the vacuum, which is renewed with each step.

In general, liners of the present invention may comprise an elastic or elastomeric fabric 54 which overlays all or a portion of the sidewalls 16 of the liner body. Such fabrics can have an elasticity which is greater than or less than the elasticity of the liner body. In some embodiments of the present invention, the liner comprises elastic or elastomeric fabrics to improve the fit of the liner and affect the overall elasticity of the liner.

It should be noted that the pin 40 of the present invention can be used with both trans-femoral and trans-tibial amputees. It could be expected that in order to tailor the effectiveness of the inventive liner product and method to a particular circumstance, it may be expedient for one of skill in the art to locate the sweat port/pin at a location other than the distal apex in order to facilitate an efficient evacuation of perspiration. In general, because of the physical parameters involved in ambulation, it is expected that the location of the sweat port would not be more than an inch from the distal apex, and in all likelihood, a smaller distance. A liner having a sweat port which is relocated to meet the perspiration drainage needs of a particular wearer does not represent a departure from the teachings herein and is encompassed within the scope of the present invention.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A prosthetic liner comprising:
an open upper end;
a closed bottom end incorporating a sweat port having a one-way valve at a distal tip attached to a cylindrically symmetrical buttress;
fabric sidewalls having a thickness wherein said sidewalls further comprise an inner layer of thermoplastic material having a Young's Modulus stress force value of less than 2.4 N per square millimeter containing no additives, said inner layer of thermoplastic material formed by sandblasting a mandrel prior to molding said thermoplastic material into said inner layer and further having a thermoplastic material thickness wherein said thermoplastic material has microcraters having a depth between 0.0100 and 0.0195 millimeters; and
at least one channel in the distal end of the liner which penetrates the inner layer of thermoplastic material and the cylindrically symmetrical buttress such that the one-way valve of the sweat port can conduct air, gases, and perspiration from the inner layer of thermoplastic material to the exterior of the prosthetic liner.

2. The prosthetic liner of claim 1 wherein the thickness of the sidewalls is between 1.5 and 3 millimeters.

3. The prosthetic liner of claim 1 wherein the silicone thickness at the bottom end is between 3 and 12 millimeters.

4. The prosthetic liner of claim 1 wherein the thermoplastic material is silicone.

5. The prosthetic liner of claim 1 wherein the thermoplastic material is an elastomer gel.

6. The prosthetic liner of claim 6 wherein the elastomer gel is a styrene block copolymer.
